(19) United States
(12) Patent Application Publication
(10) Pub. No.: US 2009/0306714 A1
(43) Pub. Date: Dec. 10, 2009

(54) METHOD OF PRODUCING A SUPPORT ARTICLE, A SUPPORT ARTICLE, AND A SUPPORT DEVICE INCORPORATING THE SAME

(75) Inventor: Lee Crompton, Worcestershire (GB)

Correspondence Address: DORITY & MANNING, P.A., POST OFFICE BOX 1449, GREENVILLE, SC, 29602-1449, US

(73) Assignee: Tempus Computers Limited, Birmungham (GB)

(21) Appl. No.: 12/437,877

(22) Filed: May 8, 2009

(30) Foreign Application Priority Data
May 8, 2008 (GB) GB0808365.1

(51) Int. Cl. A61F 5/00; G06F 19/00

(52) U.S. Cl. 606/242; 700/98

(57) ABSTRACT

A method of producing a support article for part of the human body, in particular the back, comprises the steps of obtaining an array of electronically encoded points defined in 3D corresponding to points on a surface of the body part utilizing a non-contact 3D digitiser. The array of points are converted into data readable by a computer aided manufacturing device. The data is adjusted to alter the surface prior to forming the article. The support article is formed incorporating the surface from the adjusted data using the computer aided manufacturing device.

The present invention relates to a method of producing a support article, a support article and a support device incorporating the same. More particularly, the present invention relates to a method of producing a support article for a part of the body, in particular the back, a support article for the body and a body support device incorporating the article.

Chronic back pain can be a highly painful ailment that restricts a sufferer's movement and prevents the sufferer from enjoying the everyday things in life that others take for granted, such as enjoying a good night's sleep, being mobile and being able to concentrate on work or leisure activities. Any way of alleviating the sufferer's pain is highly desirable.

It is known to take castings of individual parts of the human body using, for example, plaster of paris. However, such an approach suffers from various drawbacks when applied to people suffering from back pain or other musculoskeletal injuries. Firstly, the setting reaction generates a considerable amount of heat which may, in itself, be painful. In addition, it takes some time taken for the plaster of paris to set, during which the patient must be motionless, which may be difficult to achieve, and/or in itself painful.

It is also known to attach electronically detectable markers to the human body at strategic locations that are then used to generate a “cloud” of points in 3D space. This “cloud” can then be used to build up an approximation of a part of the body. This type of acquisition of such points is time consuming, the number of points that may feasibly be measured is low, and the attaching of the points may also be painful.

To the knowledge of the applicants, no method has hitherto been devised of rapidly and cost-effectively generating a three-dimensional profile of part of the human body which can then be used to produce a support article for that part of the body.

The present invention seeks to overcome, or at least mitigate the problems of the prior art.

Accordingly, one aspect of the present invention provides a method of producing a support article for part of the human body, in particular the back, the method comprising the steps of:
a) obtaining an array of electronically encoded points defined in 3D corresponding to points on a surface of the body part utilising a non contact 3D digitiser
b) converting the array of points into data readable by a computer aided manufacturing device
c) adjusting the data to alter the surface prior to forming the article
d) forming the support article incorporating the surface from the adjusted data using the computer aided manufacturing device.

An advantage of this method is that the support article is tailored to the body part when in its relaxed state, thereby alleviating pain and discomfort.

A second aspect of the present invention provides a support article produced by the method of the set out above.

A third aspect of the present invention provides a support device for alleviating pain in a patient suffering from back pain or a musculoskeletal ailment, the support device comprising a support article having a support surface, the support surface having a shape based on the body part of the patient that is to be supported, and the article being arranged within the device to support the body part when in a reclined or partially reclined state.

Embodiments of the present invention will now be described, by way of example only, with reference to the accompanying drawings in which:

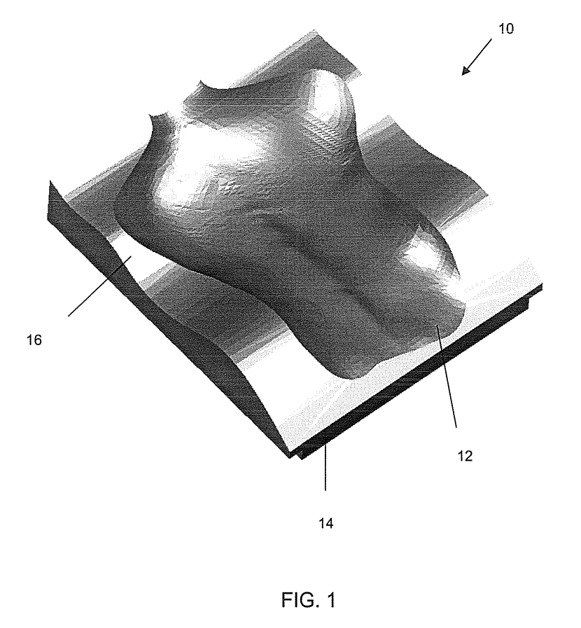

FIG. 1 is a perspective view of a support article according to an embodiment of the present invention:

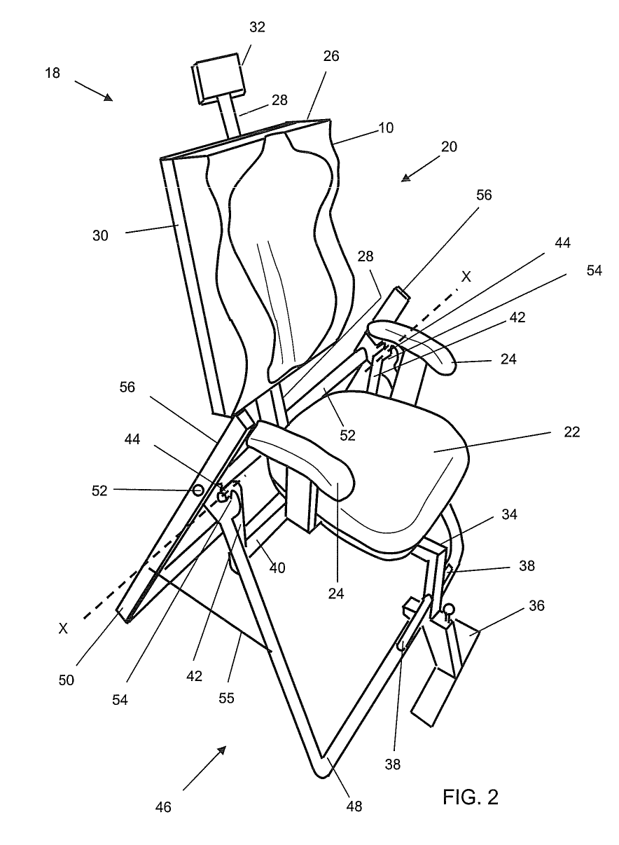

FIG. 2 is a chair incorporating a support article produced using the method of the first aspect of the present invention;

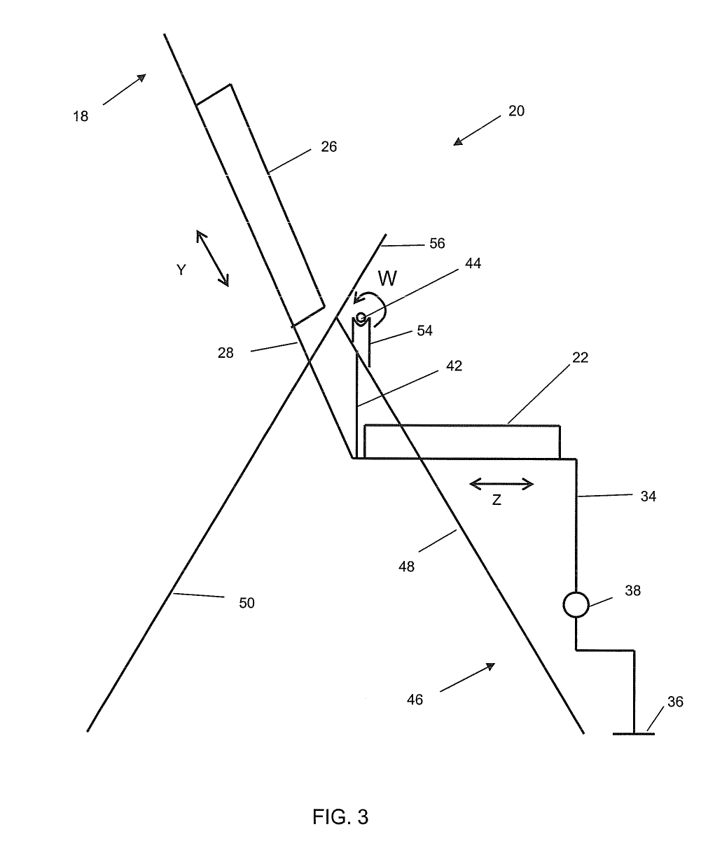

FIG. 3 is a side view of a simplified representation of the chair of FIG. 2 in an upright position;

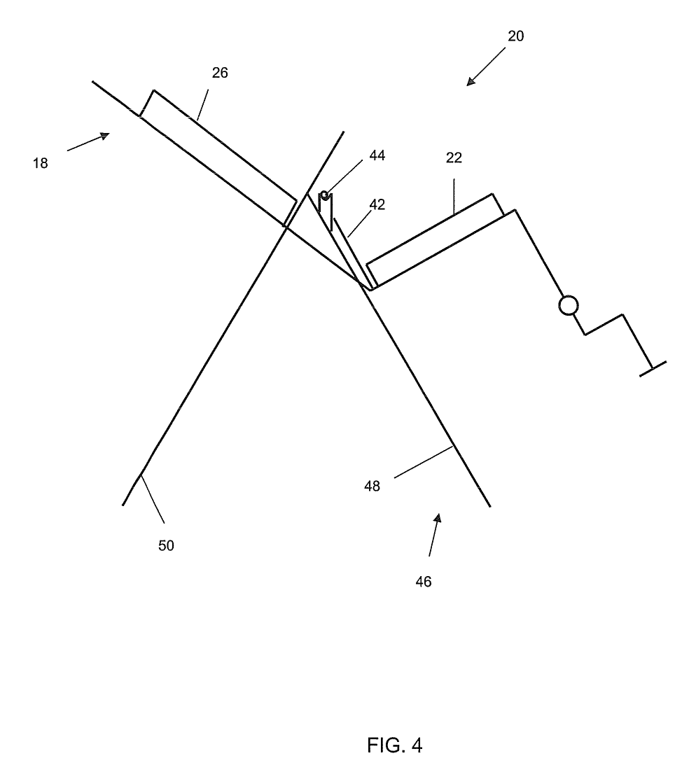

FIG. 4 is a side view of the chair of FIG. 3 in a partially reclined position;

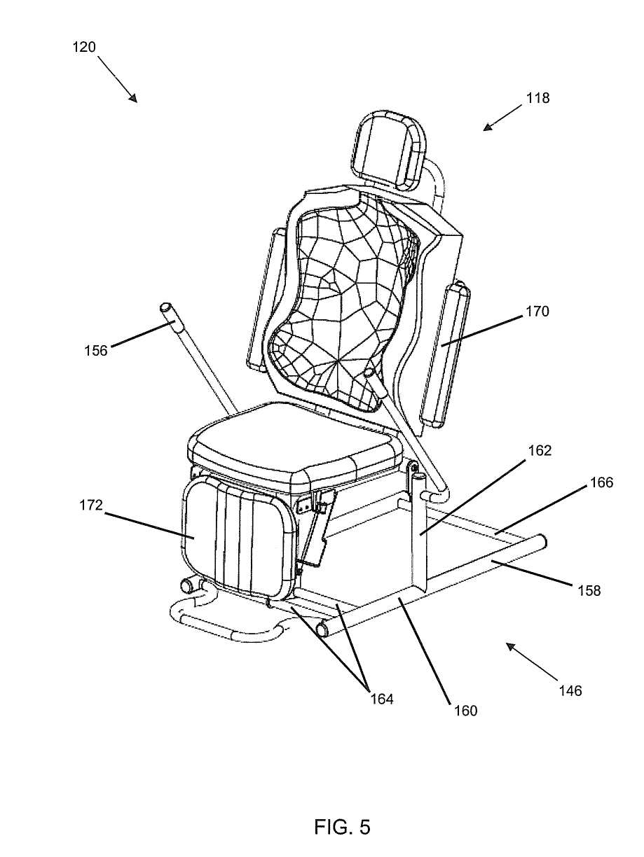

FIG. 5 is a perspective view of a second embodiment of a chair incorporating a support article produced using the method of the first aspect of the present invention in an upright position; and

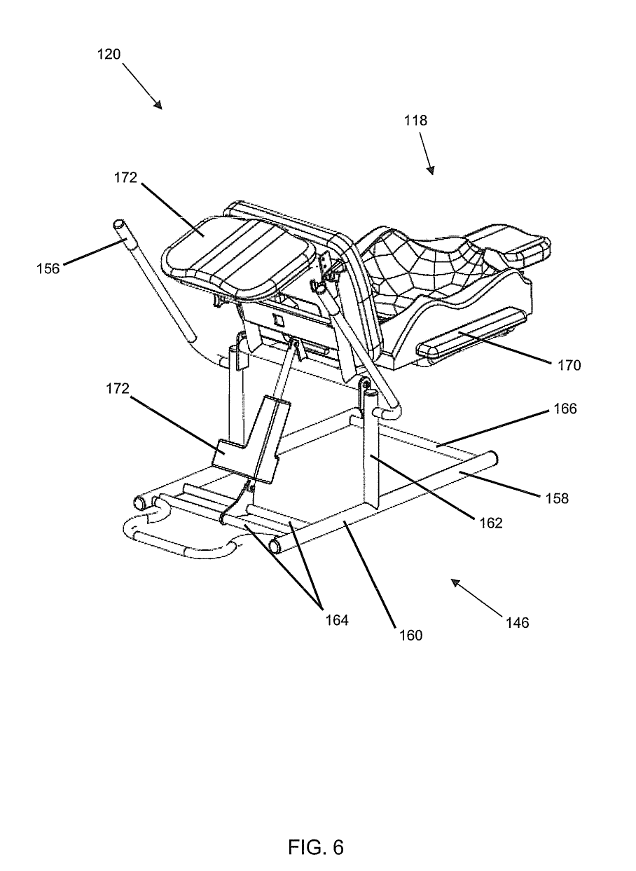

FIG. 6 is a perspective view of the chair of FIG. 5 in a fully reclined position.

Non-contact 3D digitisers use laser beam light sectioning technology to scan work pieces. The light reflected from the work piece is obtained using a digital camera. Three-dimensional data is then created by a triangulation process to determine distance information. The array of individual points in 3D space can then be joined up to build a three dimensional representation of the surface of the work piece being scanned using appropriate computer software. The scanning process is very rapid, taking three seconds or less to scan a substantial area of a work piece. Examples of such non-contact 3D digitisers are the VI-9I (known as the VIVID 9i outside Europe), the VI-910 (VIVID 910 outside Europe) and the RANGE7, all produced by Konica-Minolta. These products typically quote an accuracy of +/−less than 100 microns, indeed as low as +/−40 microns for each measured point.

The first stage of the present invention is to appropriately position the portion of a patient's body, typically their back with respect to a digitiser of the type described above and to obtain an array of electronically encoded points defined in three dimensions, corresponding to points on the surface of the back. In view of the speed of scanning, no particular fixing or bracing of the patient is needed to prevent movement, and there is no contact between the digitiser and the body. In some instances multiple scans may be required to obtain a complete set of points for the portion of the body in question, but these can be “stitched together” to form a complete array of points. The raw data from the digitiser can then be input into a suitable computer aided design (CAD) software such as Solidworks®.

It is important to ensure that the support article properly supports the back in the extended state it will be in when the patient is reclined in the chair, but the general shape of the back can only be effectively scanned when upright and the spine is compressed. Different sections of the back may compress by different amounts when upright. This being so, five zones between the nape of the neck and the lumbar region back are measured, thus allowing factors appropriate to each area of the back to be used when calculating extension. Although a larger or smaller number of zones may be used, using five has been found to produce a suitable level of accuracy.

Each zone is marked by microdots placed on the surface of the back at predetermined intervals. As the shape of the back alters according to a person's position, two scans are taken. Firstly, the patient is scanned whilst their spine is upright, allowing the general shape of the back (including the spine, ribs, shoulders etc) to be determined. In the upright position the spine is shortened because fluid exits the joints between vertebrae. The patient then lies on their side for at least around five minutes, allowing the back to settle into shape and the spine to lengthen slightly due to the fluid re-entering the joints, whereupon the second scan is taken whilst the patient remains on their side and the length of the spine found, again split into the same five zones.

Using the CAD software, the array of 3D points obtained from the digitiser can be converted by suitably skilled personnel to provide a 3D surface of the patient's back that may be manipulated within the CAD software (in particular to provide a “negative” of the surface) like any other design feature. The elongation data for each zone (as found in the second scan) are applied to the corresponding shape data found during the first scan to produce an accurate negative of the shape and length of the back. This negative surface may then be incorporated into a design for a support article that may then be manufactured.

Further attributes of the patient are also taken into account, for example their Body Mass Index (weight/height²). The higher a patient's BMI, the generally more excess weight they are carrying and so the more allowance must be made in the support article for lateral redistribution of the patient's torso when their back is supported. If a patient has an injury, material can be removed from the negative surface so that pressure is not placed on the injured area. A further allowance may be made for clothes likely to be worn in the support article but not present in the scan.

With reference to FIG. 1, the support article 10 is a block of expanded polystyrene in this embodiment. The support article 10 comprises a rear face 14 shaped to be mounted within a larger support device and a contoured front face 16. The front face 16 has a basic shape that is intended to generally reflect the curves of a person's back and therefore provide a good starting point for the finished support article without impeding the movement of the patient unduly when positioned therein. When designing the support article 10 so as to be configured exactly for the patient's back, the user of the CAD software then superimposes the support surface 12 onto the front face 16 such that the major surfaces of the patient's back are fully represented by the support surface 12 in a 3D model of the article 10.

Once this 3D model of the support article 10 has been produced in the CAD software, it may be saved in a suitable format that may be read by a computer aided manufacturing (CAM) device such as a milling or routing machine or rapid prototyping device. The CAM device then produces the article in a known manner. In the case of the support article of FIG. 1, a five axis milling machine would be required to produce both the front and rear faces of the support article 10. However, if no milling to the rear face 14 is required, then a three axis milling machine may be sufficient.

In the case of a support article 10 to be manufactured from expanded polystyrene or a similarly brittle material, a further step following the milling process is required to produce a suitably durable finished support article 10. In a preferred embodiment, the article is coated in a resin material to provide a rigid and hard-wearing outer surface. It will, however, be appreciated that other suitable coatings may be provided.

In addition, the support article 10 may be manufactured from various other materials, such as wood, plastics or metal that may be directly milled to form the required support surface. In alternative embodiments, a mould may be produced by using a milling machine, and the support article 10 produced using the mould. For example, the support article 10 may be formed from composite material such as glass fibre or carbon fibre that is laid down onto the mould. This can then be allowed to harden or cure to provide the finished support article 10.

Once the support article 10 has been produced, it is then necessary to mount it within a support device.

In FIG. 2, the support device is a chair 20 comprising a seat subassembly 18 and support subassembly 46. The seat subassembly 18 of the chair 20 comprises a conventional seat base 22 and arm rests 24, along with a back rest 26 that incorporates the support article 10. The back rest is secured relative to the seat base 22 by a support arm 28 that extends up the rear face (not visible in FIG. 2) of the back rest. The support article 10 itself is secured in a substantially rectangular mounting bracket 30 that extends around its periphery.

The support article 10 is preferably mounted within the mounting bracket 30 in a removable way, e.g. by using releasable clips or fasteners, but may alternatively be secured to the bracket using adhesive or the bracket secured thereto by the resin that has applied to the expanded polystyrene simultaneously with the application of the resin.

The support arm 28 extends upwards further to also support a head rest 32.

In preferred embodiments, the mounting bracket 30 is secured to the support arm 28 by an adjustable mounting arrangement (not shown), such as a plurality of holes in the support arm that provide alternative locations for fitting of a pin or other suitable fastener. This enables the support article to be raised and lowered with respect to the seat base 22. In other embodiments this adjustment may be motorised.

A front support leg 34 extends forward from the seat base 22 and downwardly to terminate in a base plate 36. The base plate prevents the seat from tipping forward when a user sits on it, and furthermore, when a patient stands on the base plate 36 and sits down on the seat base 22, the seat is prevented from tilting backwards.

A pair of foot rests 38 extend laterally from the support leg, approximately mid-way between the base plate 36 and the seat base 22 and are intended to be used during the reclining of the seat as discussed in more detail below. The seat further comprises a cross-member 40 that extends underneath the seat base 22 and which terminates in a pair of vertically extending bars 42 at either end thereof. The bars 42 themselves terminate at their upper end with a pair of co-axially extending pivot pins 44 that extend outwardly and are arranged about a common axis X-X.

The support subassembly 46 is in the form of an A-frame formed from tubular steel members 48 at the front thereof, and 50 at the rear. Both the front and rear tubular members 48 and 50 are substantially ‘U’ shaped and the rear member additionally comprises a cross bar 52 to provide additional strength. The members 48 and 50 are pivotably secured together at point 52 at either side of the seat (only one pivot point visible in FIG. 2). The angle between the members is maintained by tie rods 55 (only one visible in FIG. 2) that extend between the Members 48 and 50 on either side of the chair 20 and that may be adjusted to alter the height of the subassembly 46.

The front tubular member 48 additionally comprises a first and second upwardly directed pivot jaws 54 either side of the chair, and proximate to the pivot point 52. The jaws 54 are dimensioned to receive the pivot pins 44 such that the seat subassembly as a whole may pivot with respect to the support subassembly 46 about axis X-X. In this embodiment, the pivot pins 44 are held in the jaws 54 under the influence of gravity alone. However, in other embodiments suitable catches may be provided to inhibit the removal of the seat subassembly 18.

The rear tubular member 48 additionally includes handle extensions 56 to the generally upwardly directed portions thereof, that may be used by the patient to assist with the tilting of the seat sub-assembly 18.

Referring now to FIGS. 3 and 4, the reclining action of the chair 20 is shown. FIG. 3 illustrates the rest position of the chair 20. When a patient wishes to use the chair, the positions of the back rest 26 may first be adjusted as illustrated by the arrow Y, and the support leg may be extended forwards or retracted backwards as illustrated by the arrow Z. The user then seats themselves on the seat sub-assembly 18 in the usual manner such that their back fits snugly within the support surface 12.

However, in the substantially upright position illustrated in FIG. 3, the patent's spine still has to support a considerable portion of the patent's weight. Thus to relieve this need for spinal support, the patient may themselves recline the entire seat sub-assembly 18 with respect to the support sub-assembly 46. This may be achieved by the patient grasping handles 56; and gently pushing against them to allow the pivoting about axis X-X to occur as indicated by arrow W.

It will be appreciated that the geometry of the seat sub-assembly, in particular the positioning of the pivot pins 44 with respect to the back rest 26 and seat base 22 results in the chair being inherently stable to allow pivoting without an undue force being required. In addition, the dimensioning of the support sub-assembly 46 by comparison with the seat sub-assembly 18 means that in whatever position, either upright or reclined, the chair 20 remains stable. Whilst in FIG. 4 the seat sub-assembly 18 is shown in a partially reclined position, it will be appreciated that additional reclining may be possible to the point at which the back rest 26 is substantially horizontal (fully reclined).

Once in the desired reclining position, the chair 20 is preferably provided with a latching mechanism (not shown) to hold the seat sub-assembly 18 in this position.

FIGS. 5 and 6 show an alternative embodiment of the chair. This embodiment is largely similar to the previous embodiment except for the frame and arm supports. Features which are substantially the same as those of the previous embodiment are given corresponding reference numbers with the additional prefix “1”.

With reference to FIGS. 4 and 5, the second embodiment of the chair 120 has a different support subassembly 146. Rather than the A frame of the first embodiment, the support subassembly 146 is in this embodiment in the form of two inverted T-shaped frames 158, where the horizontal bars 160 of each T are joined by two cross bars 164 at the front of the base and one 166 at the rear. The seat subassembly 118 is supported by the upright shaft 162 of each inverted T 158. In this embodiment a linear actuator 168 mounted between the two front crossbars and the seat base operates the reclining mechanism of the chair 120. The linear actuator 168 comprises a DC motor with a worm gear which acts on a spindle drive system or a ball screw system.

Further differences between this and the first embodiment include the additional arm supports 170 and a lower leg support 172, all of which are mounted on the seat subassembly 118.

In a further embodiment (not shown), a pressure sensitive strip may be attached to the underside of the chair (e.g. at the rear of the lower leg support and/or at the rear of the back support). The strip is linked to a control system of the linear actuator. If pressure is exerted on the strip, for example if it is brought into contact with an object whilst the chair is being reclined or brought upright, the strip will prevent the linear actuator from further movement. This safety mechanism prevents damage to the obstruction as well as protecting the chair. A further embodiment (not shown) may have handle extensions 56, 156 attached to the seat base 22 rather than to the support subassembly 46, 146.

It has been found a considerable amount of relief from back pain has been achieved using the chairs 20 and 120 that incorporate the support articles 10 and 110. Users that have hitherto had serious problems sleeping due to constant back pain have been able to use the chair to obtain a prolonged period of sleep. In addition, the relief from pain while awake has enabled patients to undertake everyday activities such as reading or viewing the television in comfort.

Terms such as “front”, “rear”, “upper” and “lower” are used in this specification for ease of description and should not be considered limiting.

It will be appreciated that numerous changes may be made within the scope of the present invention. The support articles 10 and 110 may be incorporated into other devices such as wheelchairs, car seats or beds. The support article may be manufactured to support other parts of the body such as the neck or leas. Instead of simply reproducing the patient's back profile, the CAD software may be used to apply certain further adjustments to the profile before the manufacturing of the support article in order to provide corrective support to the patient's back that may assist the recuperation of the patient rather than simply alleviating their pain in the short term.

The chairs 20 and 120 may take numerous alternative forms and may be adjusted by using numerous alternative mechanisms. In particular, it is anticipated that the reclining motion may be motorised using various actuation mechanisms such as an electric motor driven screw meshing with a sector gear, a wire and pulley arrangement, scissor actuator, linear actuator, or the like. In addition to reclining, such a mechanism may be used to aid the patient in standing up from the chair, by lifting the chair into a more forward tilting position. The back rest of the chair may recline independently of the seat base

Additional functions such as ventilation, massaging, etc. may be incorporated into the chair. A thin layer of cushioning material may be provided over the support surface.

The order in which the two scans are taken may be reversed. If this were the case, the shape data obtained from the second scan could be used to adjust the length data found in the first scan. Once a sufficient body of data has been acquired for multiple patients it may be possible to compile a database or table of adjustment factors that can be applied to the first scan rather than performing a second scan. In order for accurate adjustments to be made enough data should be gathered from a range of people of varying age, race and sex since these factors have been found to affect the extent of elongation.

What is claimed is:

1. A method of producing a support article for part of the human body, in particular the back, the method comprising the steps of:
a) obtaining an array of electronically encoded points defined in 3D corresponding to points on a surface of the body part utilising a non contact 3D digitiser
b) converting the array of points into data readable by a computer aided manufacturing device
c) adjusting the data to alter the surface prior to forming the article
d). forming the support article incorporating the surface from the adjusted data using the computer aided manufacturing device.

2. The method of claim 1 wherein the computer aided manufacturing device is a milling machine.

3. A method according to claim 1 wherein the support article is expanded polystyrene.

4. A method according to claim 3 comprising the further step e) of coating the expanded polystyrene in resin material.

5. A method according to claim 1 comprising the further step of mounting the support article in a support device.

6. A method according to claim 1 wherein in step b) the converting of the array of points is carried out using computer aided design software.

7. A method according to claim 1 wherein the part is the back or a part of the back.

8. A method according to claim 7 wherein in step c) the data is adjusted to account for elongation of the back in a reclined position.

9. A method according to claim 8 wherein in step a) the array is obtained with the back in a substantially upright position.

10. A method according to claim 9 wherein in step c) the data is adjusted with regards to a second array obtained with the back in a recumbent position.

11. A support article formed according to the method of claim 1.

12. A support device for alleviating pain in a patient suffering from back pain or a musculoskeletal ailment, the support device comprising a support article having a support surface, the support surface having a shape based on the body part of the patient that is to be supported, and the article being arranged within the device to support the body part when in a reclined or partially reclined state.

13. A support device according to claim 12 further comprising a mechanism to enable the support article to move from a substantially upright position to the reclined or partially reclined position.

14. A support device according to claim 12 further comprising a second support surface for supporting another portion of a patient's body.

15. A support device according to claim 14 further comprising a mechanism to adjust the position of the support surface with respect to the second support surface.

16. A support device according to claim 12 wherein the support surface is positioned within the device to support the patient's back in use.

17. A support device according to claim 14 wherein the support device is a chair or wheelchair and the support surface comprises at least a portion of a back rest of a chair.

18. A support device according to claim 14 wherein the device is a chair or wheelchair and the second support surface comprises a seat base.

19. A support device according to claim 17 wherein the device is a chair or wheelchair and the second support surface comprises a seat base.

20. A support device according to claim 19 wherein the back rest pivots with respect to the seat base to recline.

21. A support device according to claim 19 wherein the back rest and seat base pivot together with respect to the remainder of the chair.
